(19) United States
(12) Plant Patent Application Publication
(10) Pub. No.: US 2024/0306521 P1
(43) Pub. Date: Sep. 12, 2024

(54) CANNABIS PLANT NAMED 'RSBT1BRM'

(50) ;

(71) Applicant: RS BioTherapeutics, Inc., Cumberland, MD (US)

(72) Inventor: Nicholas Stromberg, Sebastopol, CA (US)

(21) Appl. No.: 18/445,885

(22) Filed: Feb. 29, 2024

Related U.S. Application Data
(60) Provisional application No. 63/488,683, filed on Mar. 6, 2023.

(51) Int. Cl. A01H 6/28

(52) U.S. Cl. CPC A01H 6/28
USPC PLT/258

(57) ABSTRACT

The unique annual herbaceous Cannabis plant variety ‘RSBT1BRM is provided.

### CROSS REFERENCE TO RELATED APPLICATIONS

The present Application for Patent claims benefit of Provisional Application No. 63/488,683 entitled “CANNABIS PLANT NAMED ‘RSBT1BRM’” filed Mar. 6, 2023, which is hereby expressly incorporated by reference herein.

Latin name of the genus and species: Genus—Cannabis. Species—Hybrid.

Variety denomination: The new Cannabis plant claimed is of the variety denominated ‘RSBT1BRM’.

### BACKGROUND OF THE INVENTION

### Field of the Invention

The present invention relates to a new and distinct annual variety of Cannabis, which has been given the variety denomination of ‘RSBT1BRM’. ‘RSBT1BRM’ is intended for use as use as a medicinal herb for sale in Cannabis dispensaries, high value cannabinoid extraction and to make products containing CBD, etc.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel, and medicines.

Cannabidiol (CBD) extracted from Cannabis is widely used in over-the-counter medicines and topical treatments and is also the active ingredient in the FDA-approved drug Epidiolex®. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1;20(11):2083-2085; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res.1(1): 154-165.

Non-hemp forms of Cannabis, frequently referred to as marijuana, have been legalized for medicinal use in many states and for recreational use (sometimes called “adult use”) in a growing number of states. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

The present invention relates to a new and distinct annual variety of Cannabis, which has been given the variety denomination of ‘RSBT1BRM’.

The new Cannabis variety is a selection resulting from a sexual cross of Cannabis plants at Jacksonville, OR in 2018, involving a seed parent known as ‘Ringo's Gift’ (unpatented) x ‘Freedom Baby F2’ (unpatented) and a pollen parent known as ‘Kush Hemp’ (unpatented).

The seed lot from which ‘RSBT1BRM’ was selected was created in Jacksonville, Oregon in 2018. The initial phenotyping of it took place in Sebastopol, California during the Winter of 2022-2023. The first-generation mother block was grown in Sebastopol, California.

Asexual reproduction of the new variety has demonstrated that the new variety reproduces true to type with all of the morphological characteristics, as herein described, firmly fixed and retained through successive generations of such asexual propagation.

‘RSBT1BRM’ is different from all three lines from which it was derived. It is squatter in stature with denser flowers than ‘Ringo's Gift’, though retains its intense fruity aroma profile. Similar to the ‘Freedom Baby’ it has high flower: shoot proportion, extremely high trichome density, and relatively compact plant architecture. ‘RSBT1BRM’ owes its very high flower density to the influence of the ‘Kush Hemp’, although it has a higher flower: shoot proportion and stronger aroma than the ‘Kush Hemp’. The most similar lines to this line are from other (Harley Tsu x Freedom Baby F2) x ‘Kush Hemp’.

### BRIEF DESCRIPTION OF THE DRAWINGS

The accompanying photographic illustrations show the typical appearance of the new variety ‘RSBT1BRM’. The colors are as nearly true as is reasonably possible in a color representation of this type. Colors in the photographs may differ slightly from the color values cited in the detailed botanical description which accurately describes the colors of the new plant.

FIG. 1 is a photograph of the new variety ‘RSBT1BRM’, demonstrating compact morphology and short internodal length.

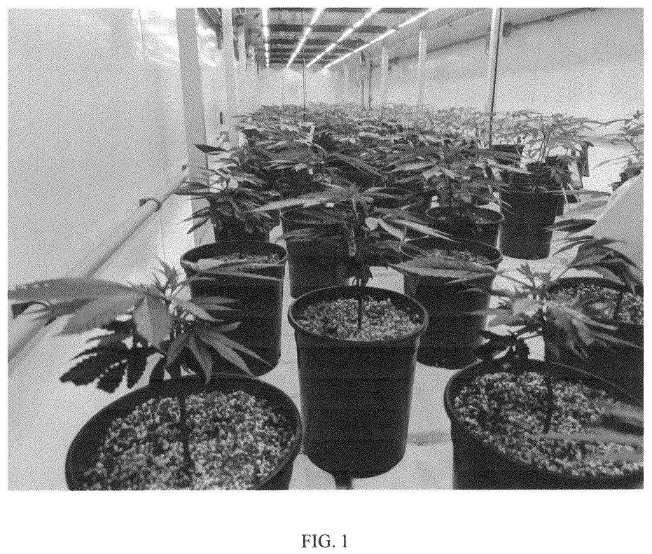

FIG. 2 is a photograph of dense capitate stalked trichomes on the new variety ‘RSBT1BRM’ at 45 days of flowering on 12/12 photoperiod.

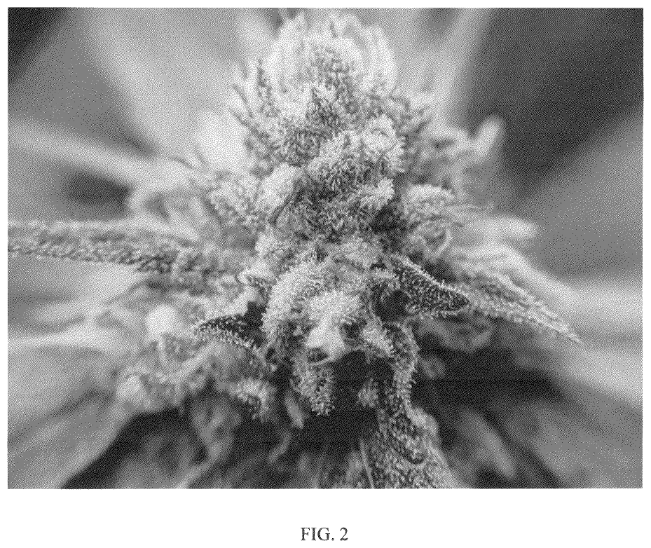

FIG. 3 is a photograph of broad leaves and early flowering of the new variety ‘RSBT1BRM’ at 25 days of generative growth.

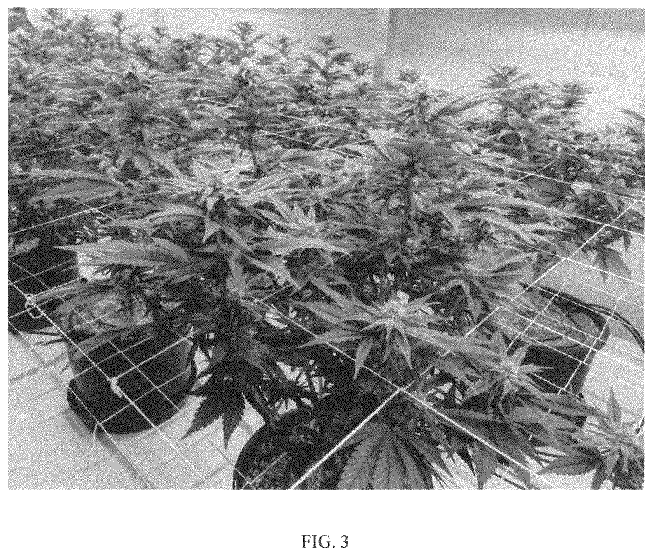

FIG. 4 is a photograph of low, well-trained canopy with tight spacing of inflorescences of the new variety ‘RSBT1BRM’ at 35 days of generative growth at 12/12 photoperiod.

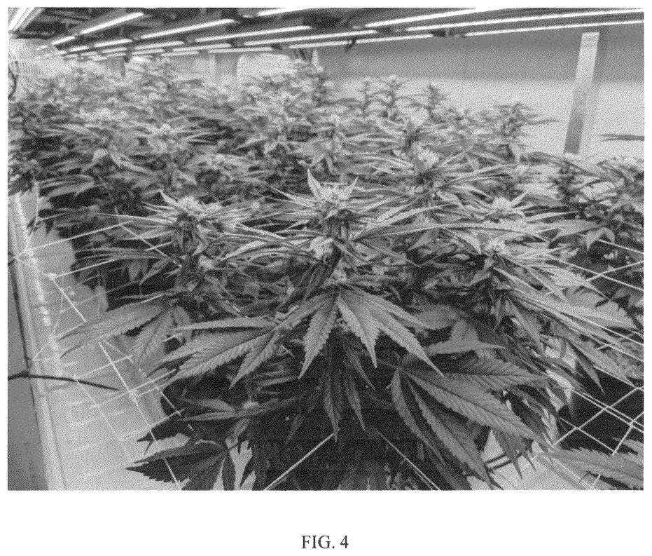

FIG. 5 is a photograph of the inflorescence continuity extending to the bottom of the canopy of the new variety ‘RSBT1BRM’ at 35 days of generative growth.

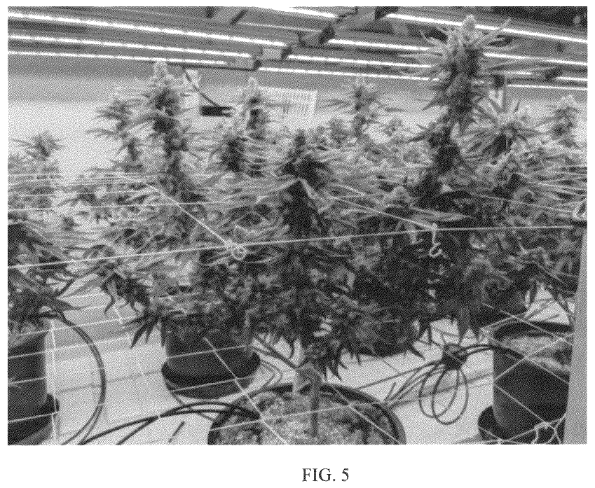

### DETAILED BOTANICAL DESCRIPTION

The following detailed description sets forth the distinctive characteristics of ‘RSBT1BRM’. The datum which defines these characteristics was collected from asexual reproductions of the original selection. Dimensions, sizes, colors, and other characteristics are approximations and averages set forth as accurately as possible. The plant history was taken on mother plants at approximately 3 weeks of vegetative growth, and the descriptions relate to plants grown in mother chamber.

Type: Herbaceous annual.

Classification:
a. Family—Cannabaceae.
b. Genus—Cannabis.
c. Species—Hybrid.
d. Common Name—Hemp.

Market Class: Hemp-derived botanical CBDa.

### PLANT

General:
   a. Parentage.—i. Female parent — ‘Ringo's Gift’ x ‘Freedom Baby F2’. ii. Male parent — ‘Kush Hemp’.
   b. Growth habit.—Compact.
   c. Hardiness.—USDA plant hardiness Zone 6-10.
   d. Average height.—0.61 meters (24″).
   e. Average spread.—0.3 meters (12″).
   f. Growth rate.—0.0081 meters (75 days/0.61 ending height).
   g. Flower storage life.—30-60 days prior processing.
   h. Productivity.—113.5 g/plant.
   i. Disease resistance/susceptibility.—Strong vigor, mild susceptibility to Erisiphales, Podosphaera/powdery mildew.
   j. Pest resistance/susceptibility.—Mild susceptibility to Polyphagotarsonemus latus, Tetranychidae.
   k. Fragrance.—Strong Kush profile featuring Cannabis sulphur compounds and earthy notes.
   l. Proportion of hermaphrodite plants.—Low.
   m. Proportion of female plants.—This plant is exclusively propagated via tissue culture or mother stock.
   n. Proportion of male plants.—None.
   o. Type of inflorescence.—Dioecious, dense, spike like.
   p. Photoperiodicity.—Short day flowering plant.

### STEM/CENTRAL STALK

General:
   a. Average diameter of stem.—1.27 cm.
   b. Average length of stem.—15.24 cm-20.32 cm.
   c. Shape.—Bushy and compact.
   d. Average length of internode.—0 to 4 cm.
   e. Depth of grooves.—Medium.
   f. Pith in cross section.—Medium.
   g. Trichome type.—Cystolithic hair, bulbous, capitate sessile and capitate stalked.
   h. Color.—Green.

### LEAF

General:
   a. Quantity.—Dense and bushy, between 20-150 depending on plant height.
   b. Leaf color (top side).—Dark green.
   c. Leaf color (under side).—Medium green.
   d. Leaf arrangement.—Opposite decussate.
   e. Leaf shape.—Compound structure with individual leaflets radiating from a central point.
   f. Leaf margins.—Serrated margins.
   g. Undulation of margin.—None.
   h. Leaf apex shape.—Acuminate.
   i. Leaf base shape.—Attenuate.
   j. Average leaf width.—15-20 cm.
   k. Average leaf length.—15-25 cm.
   l. Number of leaflets per leaf.—5-9 cm.
   m. Central leaflet length.—10-15 cm.
   n. Central leaflet width.—1-2 cm.
   o. Number of teeth/serrations in the middle of leaflet.—Dozens.
   p. Margin type.—Serrated.
   q. Trichome type (top surface).—Bulbous.
   r. Trichome type (bottom surface).—Stalked.
   s. Texture of top and bottom surfaces.—Smooth.
   t. Appearance of top and bottom surfaces.—Shiny.
   u. Venation type.—Pinnate.

### PETIOLE

General:
   a. Length.—5-10 cm.
   b. Diameter.—3 mm.
   c. Surface texture.—Slightly rough.
   d. Strength.—Medium.
   e. Anthocyanin intensity.—Not visible.
   f. Color.—Emerald to dark green.

### PEDICEL

General:
   g. Length.—3-5 mm.
   h. Diameter.—1-3 mm.
   i. Surface texture.—Slightly rough due to trichomes.
   j. Strength.—Medium.
   k. Anthocyanin intensity.—Not visible.
   l. Color.—Emerald to dark green.

### FEMALE FLOWER

General:
   a. Natural flowering season.—Late summer to early autumn.
   b. Color.—Green.
   c. Shape.—Spike.
   d. Length.—3-7 cm.
   e. Diameter.—2-6 cm.
   f. Number of petals.—No petals.
   g. Quantity per inflorescence.—None.
   h. Description when flower has bracts with no styles.—Immature flower.
   i. Description when flower has styles on the first flowers.—Pre-flower. i. Style length — Fraction of a mm. ii. Style color — White.
   j. Description when 50% of the bracts are formed.—Medium flower.
   k. Pistil.—i. Number per flower — Dozens. ii. Length — Few mm to 2 cm.
   l. General description of calyx.—Small, tear drop shape.
   m. General description of terminal inflorescence.—A spear tip.
   n. General description of node.—Slight swelling or thickening of the stem.
   o. Bract.—i. Size — 3 mm to 1 cm. ii. Quantity — Dozens. iii. Shape — Tear drop or spade shaped. iv. Trichome type — Cystolithic hair, bulbous, capitate sessile and capitate stalked. v. Color — Dark to light green. vi. Case — Bracts and calyxes. vii. Texture — Sticky and resinous. viii. Involucral bracts — 3 mm to 1 cm.
   p. Bracteole.—i. Size — 3 mm. ii. Quantity — Each flower has multiple. iii. Shape — Small, leaf like shape. iv. Trichome type — Cystolithic hair, bulbous, capitate sessile and capitate stalked. v. Color — Clear to milky.
   q. Stigma.—i. Diameter — 0.5-2 mm. ii. Quantity — Dozens. iii. Shape — Long slender strands. iv. Trichome type — Cystolithic hair, bulbous, capitate sessile and capitate stalked. v. Color — White to orange and red.
   r. Stipule.—i. Length — 1-3 cm. ii. Diameter — 0.5-2 mm. iii. Quantity — Dozens. iv. Shape — Stalk like. v. Texture — Smooth. vi. Strength — Medium. vii. Trichome type — Cystolithic hair, bulbous, capitate sessile and capitate stalked. viii. Color — Green.
   s. Ovary.—i. Position — Within the inflorescence. ii. Shape — Spear shaped. iii. Length — 1-3 mm. iv. Color — Green to white.

1. A new and distinct variety of Cannabis plant named ‘RSBT1BRM’, as illustrated and described herein.
